(19) United States
(12) Patent Application Publication
(10) Pub. No.: US 2025/0191755 A1
(43) Pub. Date: Jun. 12, 2025

(54) ARTIFICIAL INTELLIGENCE-BASED DIFFERENTIAL DIAGNOSES METHODOLOGY TO DEMARCATE DISEASE CONDITIONS HAVING OVERLAPPING CLINICAL REPRESENTATIONS

(71) Applicant: Irfanur RAHMAN, Bangalore (IN)

(72) Inventor: Irfanur RAHMAN, Bangalore (IN)

(21) Appl. No.: 18/843,179

(22) PCT Filed: Feb. 25, 2023
(86) PCT No.: PCT/IN2023/050178

§ 371 Date: Aug. 30, 2024

(30) Foreign Application Priority Data
Mar. 3, 2022 (IN) 2022410114660

(51) Int. Cl. G16H 50/20; G16H 10/20

(52) U.S. Cl. CPC G16H 50/20; G16H 10/20

(57) ABSTRACT

A method of assessing and analysing closely mimicking or overlapping patient clinical characteristics, presentations, manifestations and conditions that often appear confusing to treating physicians and medical/clinical experts, and pose concerns for true diagnostic detection of the actual underlying disease, is described and presented in here which utilizes Artificial Intelligence-based approach that requires less manual involvement and will drastically reduce diagnostic turnaround time thereby proving effective and beneficial for starting appropriate treatments quickly during unprecedented times like a pandemic when fatal fear of novel disease spread limits in-person physical examination of the affected as well as to be deployed during regular medical practices/emergencies amidst global or regional lockdowns and in other related/relatable fields.

### TECHNICAL FIELD

The present invention is aimed at assessing and analyzing the closely mimicking or overlapping (and not ‘all’ the) clinical characteristics, presentations, manifestations and conditions (signs, symptoms, and patient-derived/-generated/-reported data) of two or more diseases/diagnoses utilizing the Artificial Intelligence (AI) program to arrive at the most definitive actual diagnosis quickly with lesser manpower or manual involvement and with higher accuracy in a much shorter turnaround time especially during eras of unprecedented time like during a pandemic (when fatal fear of novel disease spread looms thereby thwarting in person physical examination of patient) or when life-saving treating modality needs to be deployed immediately in routine medical practices/emergency situations amidst global or regional lockdown scenarios and in other related/relatable fields as described in this invention application in subsequent relevant sections.

### BACKGROUND OF THE INVENTION

The unexpected outbreak of the infectious Coronavirus disease (COVID-19; caused by a newly discovered coronavirus) began gripping the unprepared World with fatal fear towards the dawn of the fourth quarter of Year 2019. With COVID-19 dominating 2020 and portions of 2021 remaining under scattered global lockdowns as precautionary measure, today even after over two years since the outbreak, in some regions of the World vaccination attempts are still in progress with a large portion of the human population yet awaiting to be immunized while some regions continue their struggle for access to vaccines. To add to our present existing woes, newer virulent and highly contagious strains continue to be discovered every now and then that plague us and are here to haunt us for a long time.

Since its outbreak, we have witnessed a lot of deaths globally-deaths that have not even spared our healthcare professionals and experts. In fact, with the ever-rising number of healthcare professionals that have already yielded to this deadly virus till date (elderly age and frontline health care profession seeming to be major predisposing factors) and with fear of tertiary and subsequent waves of new and more mutant strains impending over us, the global dearth of healthcare professionals is still so evident. Though the message of loss of lives due to COVID-19 globally is loud and obvious, there is another side to this story—which remains disregarded and often unthought-of about—i.e. the conditions which closely mimic COVID-19 disease.

There exist a myriad of other conditions that owing to their symptomatic, physiological and clinical manifestational resemblances with that of COVID-19 were mistakenly interpreted as COVID-19 itself in the above duration and hence misdiagnosed by the already overwhelmed treating physicians who were plagued with stress, tiredness, anxiety, loss of sleep, despondency and confusion coupled with being understaffed, under-resourced and having fear of exposure to COVID cases, leading to consequently losing critical patients who were essentially COVID-19-negative (i.e. not infected with COVID-19) but presented with conditions like advanced Pulmonary Tuberculosis and Acute Exacerbations of Chronic Obstructive Pulmonary Disease (COPD) and Asthma.

Whenever we hear about someone having a fever, we know that it is the body's own mechanism and first immediate innate response to combat the illness caused by an infection or the presence of foreign element/pathogen like a virus, bacterium, parasite, etc. Fever can also be caused by (i) certain inflammatory disease conditions like septic arthritis, or (ii) by conditions like a malignant tumor, or (iii) as a response to certain medications like antiepileptics, or (iv) due to introduction of live attenuated biological preparation into the body in the form of a vaccine to render acquired immunity.

So, a fever will often be seen when someone is suffering from conditions like a persistent common cold or a seasonal flu or in certain cases of worsening of few pre-existing conditions, as well as with COVID-19 infection.

While making any diagnosis or rather before arriving at a confirmatory diagnosis, the treating physician will be taking into consideration the presented signs, symptoms, clinical manifestations, presentations, duration of ailment along with other compounding factors like gender, age, sociocultural/socioeconomic backgrounds, occupational histories, already existing medical histories, any past (resolved) medical histories, known allergies, parents'/sibling(s)' medical histories, etc. Doing so, aids in performing a differential diagnosis [since one presentable symptom may be common for many diagnoses (as stated above)] thereby often offering a better clinical judgment of the actual underlying diagnosis. This in turn aims at better and robust treatment or preventative modalities.

However, often differential diagnoses itself can be subjected to risk(s) especially when symptoms tend to be common and often overlapping between closely resembling conditions having altogether different etiology thereby demanding different treatment modalities. Also, years of experience via clinical practice differs between physicians and medical/clinical experts, i.e. more experienced physicians are likely to have an edge over less experienced physicians, while some may argue that more experienced physicians being comfortable with their contemporary treatment modalities lack the openness to embrace newer treatment protocols compared to younger less experienced physicians who rely on newer generated data from newer modern studies. The differential diagnoses can be further sabotaged during times of a pandemic when the physician cannot be seeing and evaluating the patient personally due to fear of disease spreading further.

Hence, it is very imperative to distinguish “COVID” and “COVID-mimicking” conditions—at a hastened rate in an efficient manner, to save lives. Likewise, there is an alarmingly increased sense of urgency to differentiate closely mimicking conditions which often take time to detect and diagnose due to overlapping and resembling common clinical manifestations, presentations thereby prolonging the start of effective timely therapy.

Often these closely mimicking symptoms tend to camouflage the clinical judgment of the medical experts as well. We are not alien to the fact that physical distancing, masks, personal protective equipment worn by the doctors, healthcare workers and caregivers create difficulties in effective doctor-patient communication. And at times of a pandemic when the medical/clinical experts and fraternities are sleep-deprived, tired and often understaffed (not to forget the fact that they are often required to make difficult decisions involving ethical implications under such conditions) with often less chances of obtaining a second opinion from fellow colleagues or institutes who themselves are similarly overwhelmed elsewhere in another part of the world, beginning a treatment to ease off just the symptoms might prove to be risky to subject's life as sometimes the symptoms might get flared up deteriorating the actual underlying condition further rather than alleviating them. At times, such approach can even give birth to newer complications thereby causing more damage than benefit.

Each life matters—whether during an unprecedented pandemic or during regular medical practice/medical emergencies and hence we propose our invention idea based on the AI platform. The AI-based invention idea presented herein below can also be deployed in other specific areas of medical interest as described in the subsequent appropriate sections.

### OBJECTS OF THE INVENTION

This AI-based differential diagnosis methodology will accurately detect all possible diseases and medical conditions that present with closely mimicking or overlapping and resembling common clinical characteristics, manifestations and signs, symptoms, presentations which otherwise are generally difficult to differentiate thereby causing difficulty for medical and clinical fraternities around the world.

This AI-based differential diagnosis methodology will be foolproof, robust, quick and reliable option for medical and clinical fraternities around the world during global pandemic, national emergency situations as well as daily routine medical/clinical practices.

This AI-based differential diagnosis methodology will aid the global medical and clinical fraternities to make quicker decisions confidently when such difficult decisions involving ethical implications are to be made within a short time depending on patient's life-threatening conditions.

The AI-based differential diagnosis application would be installed in a device (i.e. desktop monitor, iPad, tablet, mobile devices, etc.) and then run wherein the desktop monitors, iPads, tablets and mobile devices would be serving as an input devices (for inputting the SSPMs either by typing or by using real-time voice-to-text transmission inbuilt in the program). Storage devices (external or internal of input devices) would be used for saving each such processed output generated report as a separate file/transcript for future references/audit purposes or for teaching/training purposes.

SSPMs (already mentioned before in this application) for different diagnoses sets can either be used to function as standalone for a particular disease specialty space (i.e. amongst respiratory diseases or gastrointestinal diseases or cerebrovascular diseases or cardiovascular diseases or in oncology or musculoskeletal diseases or metabolic diseases or neurological diseases or rare diseases or syndromes) or in combination for all the above disease specialty spaces simultaneously together.

SSPMs to be employed for this AI-based differential diagnosis methodology can be tweaked accordingly thereby requiring the tweaking or refurbishing of the AI algorithms further to help enable various combinations of diseases and syndromes together which can be utilized either in clinical trials for determining inclusion and exclusion criteria for appropriate patient selection or to be deployed at specific geographical locations (i.e. developing countries, etc.) upon specific needs for better screening purposes and epidemiological study conducts.

This AI-based differential diagnosis methodology can be used by end users like treating doctors, nurses, emergency medical personnel, any healthcare professional, institutions, hospitals in presence of the patient himself/herself or in presence of patient's initial test results and scan reports (i.e. in absence of patient's physical attendance) as well as by the patients themselves (i.e. in absence of medically qualified end user at the time of initial data entry) who can input the SSPMs via inbuilt voice guidance automation for real time text transmission ability provided by the AI algorithm itself.

This AI-based differential diagnosis methodology can be further trained to become an ML model that can aid in teaching the medical fraternities and students around the world further imparting knowledge for enhanced disease understanding and skillful competency in fields of respiratory, gastrointestinal, cerebrovascular, cardiovascular, oncology, musculoskeletal, metabolic diseases, neurological diseases, rare diseases and syndromes as well as in toxicology, forensics, criminology, histopathology, radiology, hematology, and biochemistry fields.

### SUMMARY OF THE INVENTION

Differentiating the diagnoses of diseases/medical conditions with closely identical/overlapping signs and symptoms using hematological, biochemical, histopathological, microbiological, and radiological investigations continue to pose challenges even to the experienced medical fraternity. The above mentioned techniques are time consuming, costly, and not often accessible everywhere except for only in major cities and capitals of few developing countries.

Till date over a period of close to 60 years there have been many important milestones in AI Development and its application in Healthcare and Medicine. Though there exist AI-based inventions and proposals that cater to a range of automatic diagnosis of disease conditions, such applications either only look for intradisciplinary differential diagnosis (like for example the case of differentiating Parkinson's Disease from other Parkinsonian disorders¹, automatic differential diagnosis of worsening heart failure²) or at arriving at a probable meaningful diagnosis considering all of the reported/manifested signs, symptoms³. However, till date no solution exists to warrant differentiating COVID Infections from other COVID-mimicking conditions that often present with COVID-like symptoms but are not COVID Infection in itself. Also, during the course of differentiating COVID-19 disease from other COVID-19-mimicking conditions, time is the most essential factor which certainly is not a luxury one has during pandemic situations unlike other normal times when one has lots of time to assess, evaluate and then come to a conclusion on the final diagnosis.

As this described invention methodology is based on AI platform, it will be using algorithms for helping to assess and analyse closely mimicking or overlapping patient clinical characteristics, presentations, manifestations, and conditions in order to arrive at the most definitive actual diagnosis.

Our idea additionally aims at training the AI-based ML model with the globally existing plethora of medical data already at our disposal to help the end user (be it treating doctors/nurses/emergency medical personnel/any healthcare professional/institutions/hospitals, etc.) to be in a position to identify the most probable diagnosis much early when identical/overlapping diagnostic presentations are posing a challenge for further management irrespective of an unprecedented pandemic (where in-person physical examination is a fatal risk) or geographical constraints (in cases of lockdowns wherein movement between nations and between provinces of the same nation are restricted).

The AI algorithms are intended to be taking into account only the signature signs, symptoms, clinical presentations, manifestations and not all the known signs, symptoms in order to help in pattern recognition which in turn is compared with disease-specific pattern characteristic of a particular disease.

Since there exist many closely mimicking conditions like the ones mentioned above (i.e. Tuberculosis, Acute Exacerbations of COPD and Asthma having manifestational resemblances with COVID) and the ones that would be described further in this invention proposal in subsequent claims being made, the AI algorithms might need to be tweaked and sometimes refurbished in order to cater to the requirements of differentiating closely overlapping symptoms of two or more than two diseases in order to know the most probable disease condition with high rate of accuracy based on rank ordering decision tree approach.

The back-end AI algorithms would be provided to the health care providers in the form of a front-end interface (i.e. web-based, mobile-based application, etc.) which will be used to input patient-reported/-derived data that will serve as the first and basic step to begin the whole differential diagnosis methodology.

Now, since the whole idea of this proposed invention model is to help differentiating one disease condition from the other which present with common overlapping characteristics, with each patient input/entry, a list of probable conditions would get displayed as probable diagnoses which in turn get further narrowed down (or trimmed/funneled) with additional patient inputs till the time the health care provider/end user is left with just two or three diagnoses at the maximum. It is at this stage that the closely overlapping symptoms are scrutinized for further demarcation to arrive at the most probable diagnosis. As mentioned earlier, this invention model aims at differentiating closely resembling conditions which are often misdiagnosed or difficult to diagnose appropriately and therefore require further assessment which often is time consuming and costly. However, by asking the pertinent questions to the patient (the end users/health care providers need not be present physically in front of the patient to examine him/her and this whole process can be done via an online meeting between the two from any part of the world), appropriate decisions can be made using the AI algorithm.

I—When using a desktop computer, laptop or mobile/iPad/tablet application in presence of the patient:
1. Input of patient-specific/-reported signs, symptoms, presentations, manifestations (SSPMs) in relevant input fields.
   Each SSPM is a single one time unique entry.
   A minimum of 4-5 SSPMs are needed to be entered/inputted.
   Each separate additional SSPM can be added using a relevant “next” command.
      Unless considered standard (a global standardized list of known diseases/diagnoses/disorders/syndromes will already be embedded into this AI program), the AI algorithm would suggest a better suited clinical/medical terminology each time the end user enters/types an SSPM (either in form of single word, words, phrase). Example—if the end user enters/types ‘high body temperature’ OR ‘warm body temperature’ OR ‘hot body temperature’ OR ‘burning body’, etc. on similar lines, automatically the AI algorithm would suggest “fever” to be the main SSPM.
      It is left to the end user to decide and confirm if “fever” should be selected as the SSPM (this can be done by selecting either “Yes” or “No” option from the prompt that would pop up for the question “Did You Mean?”) or he/she can even ignore the AI algorithm's suggestion to retain the original input as is entered by the end user (as a single word or words or phrase) if the end user considers their input(s) (based on patient reporting) much more specific. However, in both cases, the AI algorithm will be treating ‘high body temperature’ OR ‘warm body temperature’ OR ‘hot body temperature’ OR ‘burning body’ as “fever” itself since it follows the embedded medical logic to determine the inputted SSPM's medical relevance.
      However, when the input SSPM is not clear in case of wrong/incorrect spellings, mistyping or due to other reasons like improper character spacing while inputting/typing, etc., the AI algorithm would be prompting the end user for rechecking the inputted SSPM before prompting further to advance to the next step. Alternatively, the AI algorithm would even be suggesting SSPM based on initial alphabetical letter entries/typing as is often seen is world renowned search engines.
      The AI algorithm would even be having the functionality to translate the inputted SSPM from one language (say ‘non-English’) to another standard language (i.e. ‘English’) for uniformity of the outcome report generation and global acceptance. The standard language choice can be fixed at the beginning of the input process (i.e. ‘the first and basic step stage’) as a standard depending on country or geographical location of usage/operation.
   The moment each SSPM is inputted and accepted (i.e. the input is selected/chosen as/settled for “fever” based on the above example), the AI algorithm would now be prompting subsequent sub-input boxes/fields specific to each of the entered SSPM in order to basically help in determining the nature of and characterizing the inputted SSPM further.
      Sub-input boxes/fields are aimed at collecting additional relevant details like duration of fever, nature of the fever, time of onset of the fever (i.e. fever throughout the day vs. fever during daytimes vs. fever during nights, etc.) and other similar attributes in case “fever” is the SSPM (for all other SSPMs, depending on the SSPMs entered, relevant subsequent sub-input boxes/fields would get generated). This step is important since, as explained earlier, “fever” (for example) being common in majority abnormal conditions/diseases, it alone is not enough in order to reach the underlying diagnosis unless the nature of the fever reported is deciphered and explored further. Similar is the case with “cough” entered as SSPM; the sub-input boxes/fields need to capture if cough is with or without wheezing, if the cough is loud/audible, etc.
      A minimum of 4-5 SSPM inputs are needed for a robust probable outcome generation. Inputs greater than these are welcome since it gives more data for a better decision making compared to lesser inputs. Inputs lesser than 4-5 SSPMs might not be effectively reliable even if the first 3 (i.e. lesser than 4-5 SSPMs) SSPMs inputted are absolutely unique to a specific disease condition because doing so would introduce an element of biasedness towards one specific disease and might pose a concern in case the patient is suffering simultaneously from 2 different diseases which have different etiologies but present with overlapping symptoms.
      For cases wherein further relevant information pertaining to an entered SSPM is not available—implying no data is present to be entered for subsequent sub-input boxes/fields specific for the entered SSPM, in such cases the end user can return to inputting subsequent SSPMs.
2. The generated output report will certainly be a narrowed down highly probable diagnosis and will be effectively differentiated from a doubtful diagnosis which was causing confusion due to overlapping clinical representations.
3. The above output report generated within a short period of time based on questioning patient either directly in person or over telephonic line (in which case, the call records/verbal transcripts will be recorded and saved duly by the AI program for documentation as well as further/future references) or via online consultation (in which case, the online call/meeting will also be recorded and saved as transcripts for revisiting it back again) can now be considered for further foolproof analyses like ordering relevant scans pertinent to the most probable disease outcome identified rather than going ahead with ordering a battery of tests on more than one specific diagnosis.

II—When using a desktop computer, laptop, or mobile/iPad/tablet application in presence of only patient's initial test results and scan reports in the absence of the patient's physical attendance:
1. There might be times and situations when patient is not physically present for being questioned (either in person or over telephone or over online meeting/consultation) but patient scan results and laboratory assessment reports are available (since these can be easily faxed for second opinions) which are inconclusive or unable to help narrow down to a particular diagnosis or unable to rule out compounded diagnoses due to overlapping symptoms or in cases wherein the scan results and laboratory reports are negative or normal thereby indicating that the health concern is somewhere else.
   The scans (MRI, CT, PET/CT, Doppler, etc.) themselves also can be directly uploaded for the embedded AI program to assess further the scan outcome in cases of requirement of a secondary opinion to confirm the initial findings.
2. Next, the steps described in previous Section titled “When using a desktop computer, laptop or mobile/iPad/tablet application in presence of the patient:” can be used from hereon. The only thing different would be the physical absence of patient but data can be inputted by the end user in form of SSPMs using the scan results or laboratory assessment reports together with the initially captured doctor/nurse notes.

III—When using voice command to input patient-reported/-generated data in absence of medically qualified end user at the time of initial data entry
1. There might be times and situations when the patient is available for in-person or online consultation, but the desired healthcare practitioner/professional/family physician/hospital staff is not available at that moment.
2. Additionally, there may arise circumstances when the patient is not confident or competent to himself/herself input SSPMs in the designated relevant boxes/fields and unable to execute the relevant subsequent commands to allow him/her for further inputs.
3. In such cases, this AI program can assist such patients by using the voice command-guided SSPM capture in the designated relevant boxes/fields. The AI program is going to be equipped with speech recognition capabilities which in real time can perform text transmission ability to auto-fill the verbally mentioned SSPMs into the designated relevant boxes/fields as well as into the sub-input boxes/fields and upon completion of entries such capture would be stored as transcript files to be shared with the desired healthcare practitioner/professional/family physician/hospital staff who can view these transcripts at a later time based on their availability.

The healthcare practitioner/professional/family physician/hospital staff can check for the SSPMs inputted as well as the data auto-filled into the sub-input boxes/fields for medical coherence and also analyze the outcome generated which would follow the course as highlighted in the Section titled “When using a desktop computer, laptop or mobile/iPad/tablet application in presence of the patient:”.

### BRIEF DESCRIPTION OF THE DRAWINGS AND PROPOSED SSPMS

For a further better and complete understanding of the present invention proposal, reference to the before mentioned explanations is now being made with accompanying drawings. However, it is to be explicitly understood that each of the figures is provided for the purpose of illustration and description only and is not intended as a definition of the limits of the present invention.

FIG. 1 shows the diagrammatic representation of the general setup of how the patient-specific data [i.e. patient-derived/-generated/-reported signs, symptoms, presentations, manifestations (SSPMs)] are to be inputted into relevant input fields using either a desktop monitor or a laptop or a mobile/iPad/tablet. A secured internet connection source (i.e. Wi-Fi) would be needed to get the relevant subject online in case he/she is not available in person at site. SSPM1, SSPM2, SSPM3, SSPM4, SSPM5, SSPM6, SSPM7, SSPM8, and so on are the different SSPMs that can be inputted depending on subject's reporting or depending on whether the end user (i.e. treating physician, nurse, emergency medical personnel, etc.) wants to split patient-specific data. The alphabets A, B, C, D, E, F, G, H, I, J, K, L and M represent examples of probable diagnoses that would get generated based on initial entry of an SSPM. This list of probable diagnoses gets further narrowed down to yield more specific diagnosis upon entry of additional SSPMs until finally the most probable diagnosis/diagnoses (i.e. only 1 or maximum 2-3) are filtered out as the final probabilities in the Outcome Report with highest accuracy depending on decision tree ranking system by the embedded AI algorithm at the backend.

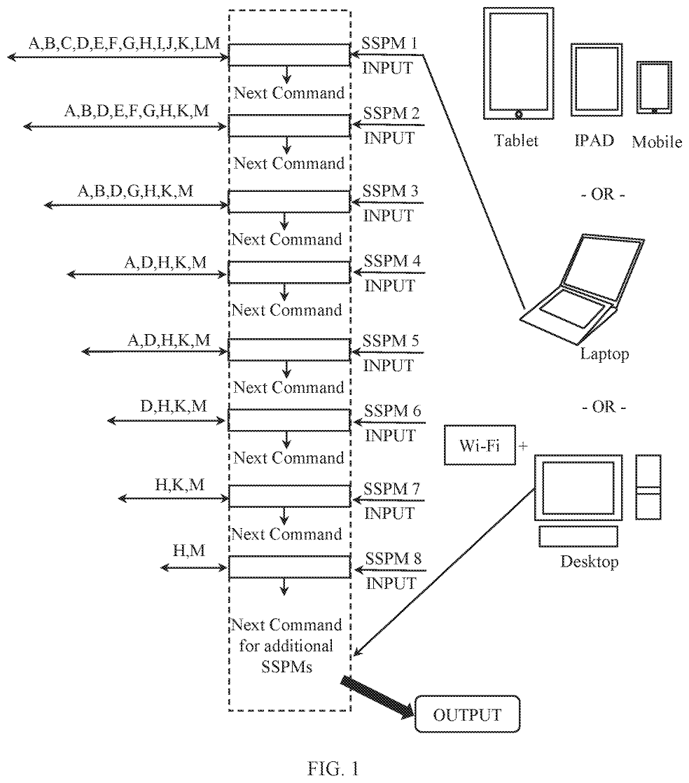

FIG. 2 details out the concept of sub-input boxes/fields that get generated upon an entry of a patient-specific SSPM by the end user. These sub-input boxes/fields further help to define the nature of each entered/typed SSPM and aid in characterization of each SSPM much more explicitly in a relevant manner. Each entered/typed SSPM will have dedicated specific sub-input box/field auto-generated that when filled/completed will prompt the auto-generation of a subsequent sub-input box/field to help further define and characterize the inputted SSPM. Any number of relevant sub-input boxes/fields can be entered/typed to arrive at a robust characterization of the inputted SSPM. At the same time when deemed prudent, any sub-input box/field can be deleted if felt not relevant or of no use. Each sub-input box/field can be added using the “next” command. When no further sub-input is to be entered, the end user can stop adding subsequent sub-input box/field and instead proceed to input the next relevant SSPM. For the next SSPM entered too, the similar format of sub-input box/field is to be followed as explained above.

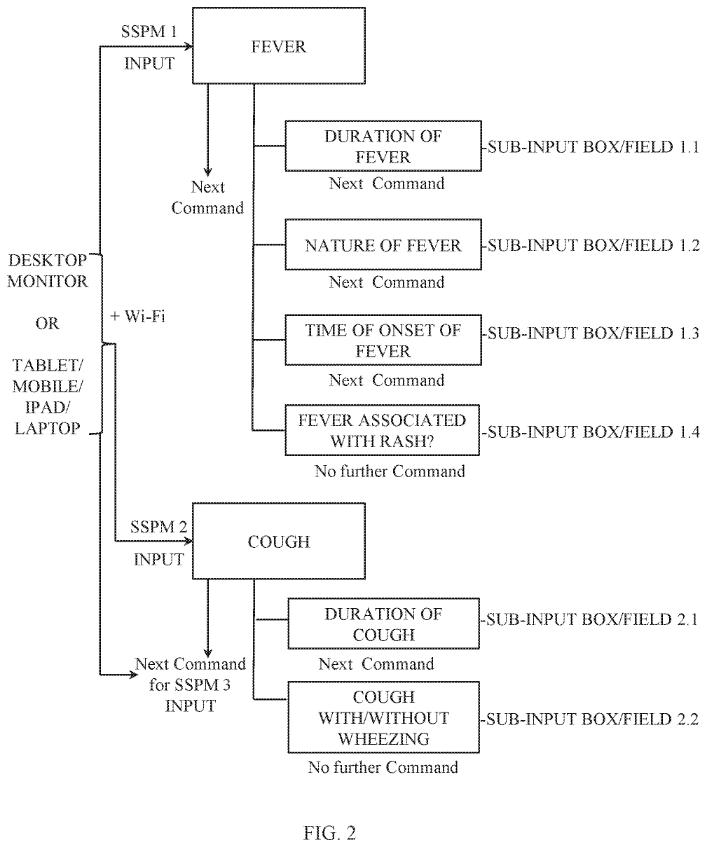

FIG. 3 details out the functionality of the AI algorithm in terms of streamlining the user-entered (i.e. patient-specific) SSPM to align with a global standardized list of known diseases/diagnoses/disorders/syndromes which already is embedded into the AI algorithm. As mentioned earlier, certain patient-reported SSPMs can be reworded or penned down more appropriately to accordingly align with medically/clinically accepted terms/nomenclatures for the ease of understanding and uniformity amongst the global medical fraternity. Therefore, keeping this requirement in mind, the embedded AI algorithm will be having this functionality to help the end user align the entered/typed SSPM with a global standardized list already embedded in order to maintain medical uniformity. The AI algorithm will be prompting a dialogue box for the end user with questions that can have either YES or NO option to be confirmed accordingly. If YES option is chosen by the end user based on AI algorithm's suggestion (since the user-entered and AI algorithm-suggested SSPM word/words/phrases from the global standardized list imply the same intention), then the user-entered SSPM gets overwritten per the AI algorithm's suggestion. In case the end user wishes to retain the initially entered SSPM despite AI algorithm's suggestion, he/she can choose NO option and the program will retain the end user's SSPM entry but will interpret it the same way as its own suggestion earlier. There will be no difference in understanding of the user-entered SSPM from the AI algorithm's suggestion since both were the same in terms of medical interpretation (as mentioned earlier, the embedded medical logic in the AI algorithm will determine the inputted SSPM's medical relevance).

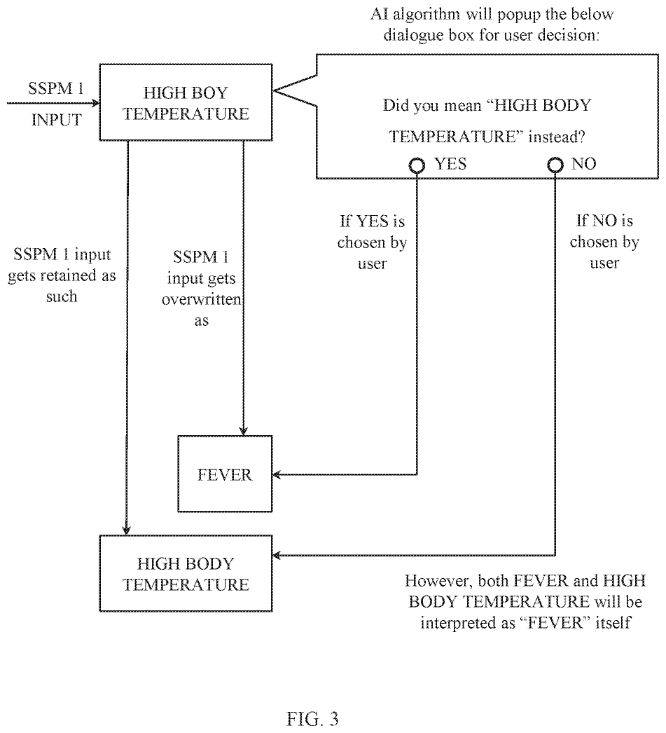

FIG. 4 illustrates the proposed AI algorithm's functionality to assess each word(s)/phrase entered by the end user as an SSPM. Cases of incorrect spellings, mistyping and even improper character spacing concerns can be picked up with this AI algorithm. Additionally, even words that appear correctly spelt per the language dictionary but have less or no relevance in terms of medical interpretation can be picked up by this proposed AI algorithm. Any time if the AI algorithm is unable to interpret the user-entered/-typed SSPM input or feels that the user-entered/typed input (in the form of word/words/phrases) does not provide much medical relevance or makes no/less medical sense due to no medical attribute associated with the inputted SSPM, the AI algorithm will interact with the end user showcasing the problem statement and suggest alternatives for the end user to check and re-assess his/her original inputted SSPM.

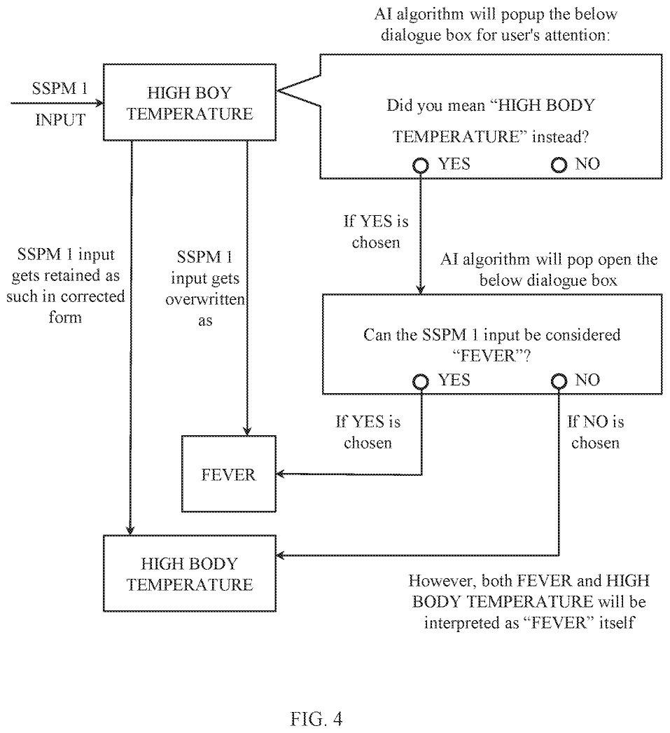

FIG. 5 showcases the functionality of the AI algorithm to translate the subject-specified end user entered/typed SSPM from native or non-standard language (example—from non-English) to a globally acceptable standard language (example—to English). This ensures uniformity while generating the outcome report for global understanding and acceptance. The inbuilt translation functionality in addition to the above described steps (described in explanations for FIGS. 1, 2, 3 and 4) simultaneously also reads the user-entered/typed input each time with no exception at all (even if entered in any non-standard language). Upon reading, the AI algorithm asks the end user if the inputted SSPM in his/her language implies the same as suggested by the AI algorithm. The AI algorithm assists the end user with the proposed standard-language synonym to be used as the entry SSPM in the standard language along with the global medically acceptable definition of the suggested terminology (in ‘standard language’ as well as in the user's ‘native or non-standard language’—the latter can be requested by the end user to the AI algorithm) which stands true for the initially entered/typed SSPM. The provided definition and the synonym in the standard language makes it easy for the end user to make the decision of accepting AI algorithm's suggestion thereby ensuring uniformity throughout the whole process of this automated differential diagnoses approach.

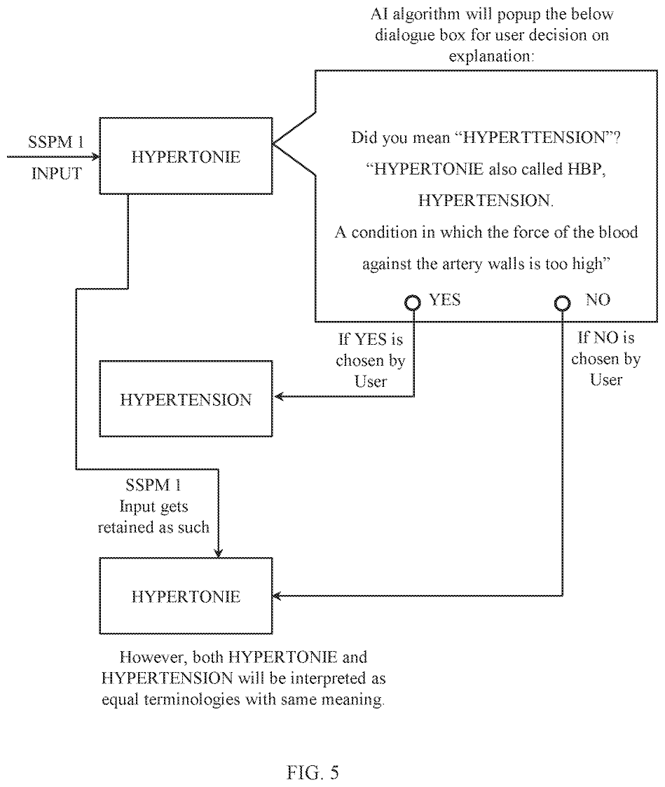

TABLE 1
Proposed SSPMs to be used for differentiating
Pulmonary Tuberculosis from COVID-19 Infection:

| SSPMs | Pulmonary Tuberculosis | COVID-19 Infection |
|---|---|---|
| SSPM 1 | Coughing for three or more weeks, | Coughing with or |
| | Coughing up blood or mucus | without wheeze |
| SSPM 2 | Fatigue | Muscle or body aches |
| SSPM 3 | Poor appetite | Poor appetite |
| SSPM 4 | Fever (Evening rise of | Fever of over |
| | temperature) with chills | 100.4° F. |
| SSPM 5 | Chest pain, or pain with | Shortness of breath |
| | breathing or coughing | |
| SSPM 6 | Unintentional weight loss | Nasal congestion |
| SSPM 7 | Night sweats | Headache and fatigue |
| SSPM 8 | | Swelling or rash on |
| | | hands and feet |
| SSPM 9 | | Loss of taste or smell |
| SSPM 10 | | Nausea/vomiting/ |
| | | diarrhoea |

TABLE 2
Proposed SSPMs to be used for differentiating
Exacerbation of COPD from COVID-19 Infection:

| SSPMS | Exacerbation of COPD | COVID-19 Infection |
|---|---|---|
| SSPM 1 | Shortness of breath (especially | Coughing with or |
| | during physical activities) | without wheeze |
| SSPM 2 | Wheezing. A chronic cough that may | Muscle or body aches |
| | produce mucus (sputum) that may be | |
| | clear, white, yellow, or greenish. | |
| SSPM 3 | Chest tightness | Poor appetite |
| SSPM 4 | Frequent respiratory infections | Fever of over |
| | | 100.4° F. |
| SSPM 5 | Lack of energy | Shortness of breath |
| SSPM 6 | Unintended weight loss (in | Nasal congestion |
| | later stages) | |
| SSPM 7 | Swelling in ankles, feet, or legs | Headache and fatigue |
| SSPM 8 | Fever | Swelling or rash on |
| | | hands and feet |
| SSPM 9 | | Loss of taste or smell |
| SSPM 10 | | Nausea/vomiting/ |
| | | diarrhoea |

TABLE 3
Proposed SSPMs to be used for differentiating
Exacerbation of Asthma from COVID-19 Infection:

| SSPMs | Exacerbation of Asthma | COVID-19 Infection |
|---|---|---|
| SSPM 1 | Shortness of breath | Coughing with or without |
| | | wheeze |
| SSPM 2 | Audible cough and wheeze | Muscle or body aches |
| SSPM 3 | Chest tightness and/or congestion | Poor appetite |
| SSPM 4 | Fatigue | Fever of over 100.4° F. |
| SSPM 5 | Not able to perform daily activities | Shortness of breath |
| SSPM 6 | Patient must stop talking to catch breath | Nasal congestion |
| SSPM 7 | No fever | Headache and fatigue |
| SSPM 8 | | Swelling or rash on hands and |
| | | feet |
| SSPM 9 | | Loss of taste or smell |
| SSPM 10 | | Nausea/vomiting/diarrhoea |

TABLE 4
Proposed SSPMs to be used for differentiating
Pneumonia from COVID-19 Infection:

| SSPMs | Pneumonia | COVID-19 Infection |
|---|---|---|
| SSPM 1 | Cough, which may produce greenish, yellow, | Coughing with or without |
| | or even bloody mucus | wheeze |
| SSPM 2 | Loss of appetite, low energy, and fatigue | Muscle or body aches |
| SSPM 3 | Fever, chills, excessive sweating | Poor appetite |
| SSPM 4 | Shortness of breath, Rapid, shallow breathing | Fever of over 100.4° F. |
| SSPM 5 | Sharp or stabbing chest pain that gets worse | Shortness of breath |
| | when you breathe deeply or cough | |
| SSPM 6 | Nausea and vomiting, especially in small | Nasal congestion |
| | children | |
| SSPM 7 | Confusion, especially in older people | Headache and fatigue |
| SSPM 8 | | Swelling or rash on hands and |
| | | feet |
| SSPM 9 | | Loss of taste or smell |
| SSPM 10 | | Nausea/vomiting/diarrhoea |

TABLE 5
Proposed SSPMs to be used for differentiating
Asbestosis from Pleural Mesothelioma:

| SSPMs | Asbestosis | Pleural Mesothelioma |
|---|---|---|
| SSPM 1 | Shortness of breath | Shortness of breath |
| SSPM 2 | Chest tightness and pain | Chest tightness and pain |
| SSPM 3 | Persistent dry cough | Persistent dry cough, Coughing up blood |
| SSPM 4 | Loss of weight and appetite | Loss of weight and appetite |
| SSPM 5 | General fatigue and weakness | General fatigue and weakness |
| SSPM 6 | Not cancerous and is limited to | Incurable cancer that develops in mesothelial |
| | the lungs and respiratory tract | tissue, typically in the lungs and abdomen. |
| SSPM 7 | Clubbing | Swelling of the face or arms |
| SSPM 8 | | Pain in the lower back or rib area |
| SSPM 9 | | Night sweats or fever |
| SSPM 10 | | Lumps under the skin on the chest |

TABLE 6
Proposed SSPMs to be used for differentiating
Pulmonary Sarcoidosis from Lung Cancer:

| SSPMs | Pulmonary Sarcoidosis | Lung Cancer |
|---|---|---|
| SSPM 1 | Fatigue | Fatigue |
| SSPM 2 | Wheezing | New onset of wheezing |
| SSPM 3 | Weight loss | Weight loss, Loss of appetite |
| SSPM 4 | Shortness of breath | Shortness of breath |
| SSPM 5 | Persistent cough | Persistent cough, Coughing up blood or rust- |
| | | coloured sputum (spit or phlegm) |
| SSPM 6 | Chest pain | Chest pain that is often worse with deep |
| | | breathing, coughing, or laughing |
| SSPM 7 | Night sweats | Hoarseness |
| SSPM 8 | Pain in the joints and bones | |
| SSPM 9 | Skin rashes, lumps, and colour | |
| | changes on face, arms, or shins | |
| SSPM 10 | Swollen lymph nodes | |
| SSPM 11 | Inflammation of the eyes and | |
| | pain, burning, blurred vision, | |
| | and light sensitivity | |
| SSPM 12 | Fever | |

TABLE 7
Proposed SSPMs to be used for differentiating
Ulcerative Colitis from Crohn's Disease:

| SSPMs | Ulcerative Colitis | Crohn's Disease |
|---|---|---|
| SSPM 1 | Abdominal pain or discomfort | Abdominal pain or discomfort |
| SSPM 2 | Bloody stools | Bloody stools |
| SSPM 3 | Cramping | Cramping |
| SSPM 4 | Constipation | Constipation |
| SSPM 5 | Overactive bowel movements | Overactive bowel movements |
| SSPM 6 | Fever | Fever |
| SSPM 7 | Loss of appetite | Loss of appetite |
| SSPM 8 | Weight loss | Weight loss |
| SSPM 9 | Abnormal menstrual cycles in | Abnormal menstrual cycles in |
| | women | women |
| SSPM 10 | Frequent diarrhoea | Frequent diarrhoea |
| SSPM 11 | Fatigue | Fatigue |
| SSPM 12 | Malnutrition | Malnutrition |
| SSPM 13 | Weight loss | Weight loss |
| SSPM 14 | Fistulas | Urgency of bowel movement |
| SSPM 15 | Skin conditions | |
| SSPM 16 | Joint pain | |

TABLE 8
Proposed SSPMs to be used for differentiating
Appendicitis from Intestinal Obstruction:

| SSPMs | Appendicitis | Intestinal Obstruction |
|---|---|---|
| SSPM 1 | Abdominal pain | Crampy abdominal pain that comes and |
| | | goes |
| SSPM 2 | Nausea and vomiting | Vomiting |
| SSPM 3 | Loss of appetite | Loss of appetite |
| SSPM 4 | Constipation or diarrhoea | Constipation |
| SSPM 5 | Abdominal bloating | Abdominal bloating |
| SSPM 6 | Flatulence | Inability to have a bowel movement or pass |
| | | gas |
| SSPM 7 | Low-grade fever that may worsen | |
| | as the illness progresses | |
| SSPM 8 | Abdominal pain | |
| SSPM 9 | Nausea and vomiting | |
| SSPM 10 | Loss of appetite | |

TABLE 9
Proposed SSPMs to be used for differentiating Stroke (Cerebrovascular
Accident) from Multiple Sclerosis (MS):

| | Stoke (Cerebrovascular | |
|---|---|---|
| SSPMs | Accident) | Multiple Sclerosis (MS) |
| SSPM 1 | Confusion | Confusion |
| SSPM 2 | Dizziness | Dizziness |
| SSPM 3 | Headache | Headache |
| SSPM 4 | Numbness | Numbness |
| SSPM 5 | Speech problems | Speech problems |
| SSPM 6 | Sudden trouble walking, dizziness, | Sudden trouble walking, dizziness, loss of |
| | loss of balance, or lack of | balance, or lack of coordination. |
| | coordination. | |
| SSPM 7 | Vision problems | Vision problems |
| SSPM 8 | Paralysis | Bladder issues |
| SSPM 9 | | Sexual dysfunction |
| SSPM 10 | | Cognitive problems |

TABLE 10
Proposed SSPMs to be used for differentiating
Atrial Fibrillation from Atrial Flutter:

| | Atrial | Atrial |
|---|---|---|
| SSPM Parameter | fibrillation | flutter |
| SSPM 1: Rapid pulse rate | Usually rapid | Usually rapid |
| SSPM 2: Irregular pulse | Always irregular | Can be regular |
| | | or irregular |
| SSPM 3: Dizziness or Fainting | Yes | Yes |
| SSPM 4: Palpitations | Yes | Yes |
| SSPM 5: Shortness of breath | Yes | Yes |
| SSPM 6: Weakness or Fatigue | Yes | Yes |
| SSPM 7: Chest pain or Tightness | Yes | Yes |
| SSPM 8: Increased chance of | Yes | Yes |
| blood clots and stroke | | |

TABLE 11
Proposed SSPMs to be used for differentiating Pericarditis from Costochondritis:

| SSPMs | Pericarditis | Costochondritis |
|---|---|---|
| SSPM 1 | The pain usually occurs behind the | Occurs on the left side of your |
| | breastbone or in the left side of your | breastbone |
| | chest. | |
| SSPM 2 | It often gets worse when you cough, | Worsens when you take a deep breath or |
| | lie down, or take a deep breath. | cough |
| SSPM 3 | Sitting up and leaning forward makes | Is sharp, aching or pressure-like |
| | you feel better. | |
| SSPM 4 | Abdominal or leg swelling | Affects more than one rib |
| SSPM 5 | Cough | |
| SSPM 6 | Fatigue or general feeling of | |
| | weakness or being sick | |
| SSPM 7 | Low-grade fever | |
| SSPM 8 | Palpitations | |
| SSPM 9 | Shortness of breath when lying down | |

TABLE 12
Proposed SSPMs to be used for differentiating Pancreatic
Cancer from Primary Gastric Lymphoma:

| SSPMs | Pancreatic Cancer | Primary Gastric Lymphoma |
|---|---|---|
| SSPM 1 | Abdominal and/or back pain | Abdominal and/or back pain |
| SSPM 2 | Low appetite and weight loss | Low appetite and weight loss |
| SSPM 3 | Nausea and vomiting | Nausea and vomiting |
| SSPM 4 | Fatigue | Fatigue |
| SSPM 5 | Diarrhoea or constipation | Constipation |
| SSPM 6 | Indigestion | Anaemia |
| SSPM 7 | Jaundice | |
| SSPM 8 | Diabetes | |
| SSPM 9 | Pale grey or fatty stool | |

TABLE 13
Proposed SSPMs to be used for differentiating Lupus from Fibromyalgia:

| SSPMs | Lupus | Fibromyalgia |
|---|---|---|
| SSPM 1 | Pain | Pain |
| SSPM 2 | Cognitive issues | Cognitive issues |
| SSPM 3 | Fatigue | Fatigue |
| SSPM 4 | Headaches | Headaches |
| SSPM 5 | Painful, swollen joints | Widespread musculoskeletal pain all over |
| | | the body |
| SSPM 6 | Swelling around the eyes and | Tension or migraine headache |
| | extremities | |
| SSPM 7 | Unintentional weight loss (or | Weird body sensations |
| | sometimes gain due to swelling) | |
| SSPM 8 | Rashes and skin lesions | Irritable bowel symptoms, pelvic pain, and |
| | | jaw/facial pain. |
| SSPM 9 | Sensitivity to sunlight and cold | |
| | temperatures | |

TABLE 14
Proposed SSPMs to be used for differentiating
Wilson's Disease from Parkinson's Disease:

| SSPMs | Wilson's Disease | Parkinson's Disease |
|---|---|---|
| SSPM 1 | Problems with speech, | Speech changes. Patient may speak softly, |
| | swallowing or physical | quickly, slur or hesitate before talking. The |
| | coordination | speech may be more of a monotone rather |
| | | than have the usual inflections. |
| SSPM 2 | Uncontrolled movements or | Tremor. A tremor, or shaking, usually |
| | muscle stiffness | begins in a limb, often your hand or fingers. |
| | | Patient may rub your thumb and forefinger |
| | | back and forth, known as a pill-rolling |
| | | tremor. Hand may tremble when it's at rest. |
| | | Rigid muscles. Muscle stiffness may occur |
| | | in any part of your body. The stiff muscles |
| | | can be painful and limit your range of |
| | | motion. |
| SSPM 3 | Fatigue, lack of appetite or | Slowed movement (bradykinesia). Over |
| | abdominal pain | time, Parkinson's disease may slow the |
| | | movement, making simple tasks difficult |
| | | and time-consuming. |
| SSPM 4 | A yellowing of the skin and the | Impaired posture and balance. Your posture |
| | whites of the eye (jaundice) | may become stooped, or you may have |
| | | balance problems because of Parkinson's |
| | | disease. |
| SSPM 5 | Golden-brown eye discoloration | Writing changes. It may become hard to |
| | (Kayser-Fleischer rings) | write, and the writing may appear small. |
| SSPM 6 | Fluid build-up in the legs or | Loss of automatic movements. Patient may |
| | abdomen | have a decreased ability to perform |
| | | unconscious movements, including |
| | | blinking, smiling, or swinging the arms |
| | | when they walk. |

TABLE 15
Proposed SSPMs to be used for differentiating
Arsenic Poisoning from Cholera:

| SSPMs | Arsenic Poisoning | Cholera |
|---|---|---|
| SSPM 1 | Nausea and vomiting | Nausea and vomiting |
| SSPM 2 | Diarrhoea | Diarrhoea |
| SSPM 3 | Muscle cramps | Muscle cramps |
| SSPM 4 | Dehydration | Dehydration |
| SSPM 5 | Electrolyte imbalance | Electrolyte imbalance |
| SSPM 6 | Abnormal heart rhythm | Irregular heartbeat |
| SSPM 7 | Red or swollen skin | |
| SSPM 8 | Abdominal pain | |
| SSPM 9 | Tingling of fingers and toes | |
| SSPM 10 | Skin changes, such as new warts or | |
| | lesions | |

TABLE 16
Proposed SSPMs to be used for differentiating Cyanide
Poisoning from Carbon Monoxide Poisoning:

| SSPMs | Cyanide Poisoning | Carbon Monoxide Poisoning |
|---|---|---|
| SSPM 1 | Nausea | Nausea or vomiting |
| SSPM 2 | Confusion | Confusion |
| SSPM 3 | Headache | Headache |
| SSPM 4 | Shortness of breath | Shortness of breath |
| SSPM 5 | Weakness | Weakness |
| SSPM 6 | Loss of consciousness | loss of consciousness |
| SSPM 7 | Vertigo | Vertigo |
| SSPM 8 | Weaker, more rapid pulse, slow, | Damage to your heart, possibly |
| | irregular heart rate, cardiac arrest | leading to life-threatening cardiac |
| | | complications |
| SSPM 9 | Dilated pupils | Blurred vision |
| SSPM 10 | Coma | Permanent irreversible brain damage |
| SSPM 11 | Bright red flush | Death |
| SSPM 12 | reduced body temperature | |
| SSPM 13 | Blue lips, face, and extremities | |
| SSPM 14 | Seizure | |
| SSPM 15 | Drowsiness | |
| SSPM 16 | Death | |

It is important to mention that though the figures, their descriptions, and the usage of proposed SSPMs outlined in Tables 1 through 16 have been described explicitly in detail in line with and to depict the advantages of the invention, it should be understood that numerous modifications, replacements, and further proposals can be made herein without actually departing from the invention and logics mentioned. With that said, it should be further borne in mind that the scope of the current invention application is not to be considered in any manner limited to the embodiments of idea, logic, steps, processes, advantages, and applicability. The invention idea described herein can be used for vast medical expanse in fields of respiratory, gastrointestinal, cerebrovascular, cardiovascular, oncology, musculoskeletal, metabolic diseases, neurological diseases, rare diseases and syndromes as well as in toxicology, forensics, criminology, histopathological, radiological, haematological, and biochemical space in times of a global pandemic, national emergency or during regular medical crisis when fear of disease spread curbing the physical examination of the patient, struggle in obtaining secondary opinion from elsewhere in the world as well as time constraint to take important appropriate decisions pertaining to life-threatening situations exist.

| Sl. | Patent | Publication | | Date of | |
|---|---|---|---|---|---|
| No. | Number | Number | Filing Date | Patent | Inventors |
| 1. | 10,980,899 | 20190328909 | 2017 Dec. 20 | 2021 Apr. 20 | Jones P A, et. al. |
| 2. | | WO 2015/175207 | 2015 Apr. 28 | | Zhany Y, et. al. |
| | | A1 | | | |
| 3. | U.S. Pat. No. | | 2013 Jul. 23 | 2017 Jan. 3 | Kabir A A |
| | 9,536,051 B1 | | | | |

1.-27. (canceled)

28. An Artificial Intelligence (AI)-based medical screening method for determining a most-specific medical condition from one or more closely mimicking medical conditions, the method is characterized to:
receiving, at an electronic device, a plurality of clinical signs, symptoms, presentations, and manifestations (SSPMs) as inputs (SSPM1, SSPM2, . . . , SSPMn) representing a patient's clinical characteristics related to the medical condition which the patient is expected to be suffering from, wherein each of the SSPMs is an individual input;
generating, by the electronic device, a plurality of SSPM-related questionnaires, comprising a plurality of SSPM-specific questions, associated with each of the SSPMs (SSPM1, SSPM2, . . . , SSPMn) received, wherein each question in the plurality of SSPM-related questionnaires are predefined;
receiving, at the electronic device, a response to each of the SSPM-specific question related to the plurality of generated SSPM-related questionnaires;
screening, by the electronic device, all the cumulative responses received for the plurality of SSPM-specific questions related to each of the generated SSPM-related questionnaires;
determining, by the electronic device, the most-specific medical condition from the one or more closely mimicking medical conditions based on the received SSPMs (SSPM1, SSPM2, . . . , SSPMn) and screened cumulative responses for the plurality of SSPM-specific questions related to each of the generated SSPM-related questionnaires; and
generating, by the electronic device, a notification based on the determined most-specific medical condition from the one or more closely mimicking medical conditions.

29. The method as claimed in claim 1, wherein the electronic device comprises an AI-based training model trained using one or more training datasets related to a global standardized list of medical conditions related to a plurality of disease spaces.

30. The method as claimed in claim 1, wherein each of the SSPMs (SSPM1, . . . , SSPMn) comprises at least one of:
an audio input related to the patient's clinical characteristics, wherein the audio input comprises at least one of: an audio call record, and/or verbal transcript;
a visual input related to the patient's clinical characteristics, wherein the video input comprises at least one of a video call record, and/or real time interaction with a medical practitioner; and
a scripted input related to the patient's clinical characteristics, wherein the transcript input comprises laboratory assessment reports comprising imaging inputs.

31. The method as claimed in claim 3, wherein the imaging input comprises at least one of: Magnetic Resonance Imaging (MRI) scan, Computed Tomography (CT) scan, Positron Emission Tomography (PET) scan, CT/PET, and Doppler scan.

32. The method as claimed in claim 1, wherein the screening comprises: differentiating and detecting, in the respiratory disease space, a ‘Pulmonary Tuberculosis’ from the COVID-19 Disease.

33. The method as claimed in claim 1, wherein the screening comprises: differentiating and detecting, in the respiratory disease space, an ‘Exacerbation of COPD’ from the COVID-19 Disease.

34. The method as claimed in claim 1, wherein the screening comprises: differentiating and detecting, in the respiratory disease space, an ‘Exacerbation of Asthma’ from the COVID-19 Disease.

35. The method as claimed in claim 1, wherein the screening comprises: differentiating and detecting, in the respiratory disease space, a ‘Pneumonia’ from the COVID-19 Disease.

36. The method as claimed in claim 1, wherein the screening comprises: differentiating and detecting, in the respiratory disease space, a condition of an ‘Asbestosis’ from that of a ‘Pleural Mesothelioma’.

37. The method as claimed in claim 1, wherein the screening comprises: differentiating and detecting, in the respiratory disease space, a condition of a ‘Pulmonary Sarcoidosis’ from the one or more closely mimicking medical conditions relating to a ‘Lung Cancer’.

38. The method as claimed in claim 1, wherein the screening comprises: differentiating and detecting, in the gastrointestinal disease space, a condition of an ‘Ulcerative Colitis’ from that of a ‘Crohn's Disease’.

39. The method as claimed in claim 1, wherein the screening comprises: differentiating and detecting, in the gastrointestinal disease space, a condition of an ‘Appendicitis’ from that of an ‘Intestinal Obstruction’.

40. The method as claimed in claim 1, wherein the screening comprises: differentiating and detecting, in the cerebrovascular disease space, a condition of a ‘Stroke’ (Cerebrovascular accident) from that of a ‘Multiple Sclerosis (MS)’.

41. The method as claimed in claim 1, wherein the screening comprises: differentiating and detecting, in the cardiovascular disease space, a condition of an ‘Atrial Fibrillation’ from that of an ‘Atrial Flutter’.

42. The method as claimed in claim 1, wherein the screening comprises: differentiating and detecting, in the cardiovascular disease space, a condition of a ‘Pericarditis’ from that of a ‘Costochondritis’.

43. The method as claimed in claim 1, wherein the screening comprises: differentiating and detecting, in the cancer biology space, a condition of a ‘Pancreatic Cancer’ from that of a ‘Primary Gastric Lymphoma’.

44. The method as claimed in claim 1, wherein the screening comprises: differentiating and detecting, in a musculoskeletal disease space, a condition of ‘Lupus’ from that of a ‘Fibromyalgia’.

45. The method as claimed in claim 1, wherein the screening comprises: differentiating and detecting a ‘Wilson's Disease’ from ‘Parkinson's Disease’.

46. The method as claimed in claim 1, wherein the screening comprises: differentiating and detecting, in the toxicology space, a condition of an ‘Arsenic Poisoning’ from that of a ‘Cholera’.

47. The method as claimed in claim 1, wherein the screening comprises: differentiating and detecting, in the toxicology space, a condition of a ‘Cyanide Poisoning’ from that of a ‘Carbon Monoxide Poisoning’.

48. The method as claimed in claim 1, wherein the screening comprises: differentiating and detecting, in a forensic and criminology space, a ‘Culpable Homicide’ from a ‘Murder’.

49. The method as claimed in claim 1, wherein the screening comprises: differentiating and detecting, a condition of a syndrome from the one or more closely mimicking medical conditions relating to rare diseases.

50. The method as claimed in claim 1, wherein the screening comprises: differentiating and detecting the one medical condition from other closely mimicking medical conditions having identical histopathological features.

51. The method as claimed in claim 1, wherein the screening comprises: differentiating and detecting the one medical condition from other closely mimicking medical conditions having identical radiological features.

52. The method as claimed in claim 1, wherein the screening comprises: differentiating and detecting the one medical condition from other closely mimicking medical conditions having identical hematological features.

53. The method as claimed in claim 1, wherein the screening comprises: differentiating and detecting the one medical condition from other closely mimicking medical conditions having identical biochemical features.
